(19) United States
(12) Patent Application Publication
(10) Pub. No.: US 2025/0043325 A1
(43) Pub. Date: Feb. 6, 2025

(54) FUCOSIDASE MUTANTS AND THE USE THEREOF

(71) Applicant: CHO PHARMA, INC., TAIPEI CITY (TW)

(72) Inventors: WEI-SHEN WU, TAIPEI CITY (TW); KUO-CHING CHU, TAIPEI CITY (TW)

(73) Assignee: CHO PHARMA, INC., TAIPEI CITY (TW)

(21) Appl. No.: 18/777,939

(22) Filed: Jul. 19, 2024

Related U.S. Application Data
(60) Provisional application No. 63/529,439, filed on Jul. 28, 2023.

(51) Int. Cl. C12P 21/00; C12N 9/24

(52) U.S. Cl. CPC C12P 21/005; C12N 9/2402; C12Y 302/01051; C12Y 302/01096

(57) ABSTRACT

The present disclosure relates to a novel mutant form of α-fucosidase (α-L-fucosidase), which exhibits enhanced α-(1,6) fucosidase activity. The present disclosure also relates to the compositions comprising the novel mutant form of α-fucosidase, and the methods of using the novel mutant form of α-fucosidase to cleave α-(1,6)-linked fucoses in the glycoconjugates.

### CROSS-REFERENCE TO RELATED APPLICATIONS

This application claims priority to U.S. Provisional 63/529,439 filed Jul. 28, 2023, which is incorporated herein by reference in its entirety.

### REFERENCE TO SEQUENCE LISTING SUBMITTED VIA EFS-WEB

This application includes an electronically submitted sequence listing in .xml format. The .xml file contains a sequence listing entitled “US14739-Sequence Listing-revised .xml” created on Aug. 5, 2024 and is 39 kilobytes in size. The sequence listing contained in this .xml file is part of the specification and is hereby incorporated by reference herein in its entirety.

### TECHNICAL FIELD

The present disclosure relates to a novel mutant form of α-fucosidase (α-L-fucosidase), which exhibits enhanced α-(1,6) fucosidase activity. The present disclosure also relates to the compositions comprising the novel mutant form of α-fucosidase and the methods of using the novel mutant form of α-fucosidase to cleave α-(1,6)-linked fucoses in the glycoconjugates.

### BACKGROUND

N-glycosylation is one of the most common posttranslational modifications observed in mammalian proteins and plays a crucial role in modulating the intrinsic characteristics and biological functions of these proteins. The attachment of N-glycans, for instance, can significantly impact protein folding, stability, antigenicity, and immunogenicity. Moreover, N-glycans can directly participate in a wide range of biological recognition processes, including cell adhesion, host-pathogen interactions, cancer metastasis, and immune responses. Although mammalian N-glycans share basic oligosaccharide core structure, the introduction of additional modifications to the core structure, such as sialylation and fucosylation, contributes to an increased level of structural diversity that effects biological functions.

The physiological activity of therapeutic antibodies is mediated by two distinct mechanisms. The first mechanism involves the target antigen neutralization or apoptosis, which contributes to the therapeutic efficacy. The second mechanism involves antibody effector functions, antibody-dependent cellular cytotoxicity (ADCC) and complement-dependent cytotoxicity (CDC), which are activated by the formation of immune complexes. The significance of ADCC in the clinical effectiveness of therapeutic antibodies, particularly in anticancer antibodies, has been elucidated through genetic analyses of leukocyte receptor (FcγR) polymorphisms in patients. Hence, the bioindustry has been spending great efforts on the development of ADCC enhancement technologies, including the modification of N-glycans attached to the constant region (Fc) of the antibody. The amount of α-(1,6)-linked fucose (core fucose) attached to the innermost GlcNAc of N-glycan on the Fc region of the antibody is known to affect the ADCC activity. Removal of core fucose from the innermost GlcNAc of N-glycan on the Fc region significantly enhances the ADCC activity of IgG because of the increased binding affinity of nonfucosylated antibodies to the FcγRIIIa receptor.

Numerous approaches have been developed to enhance FcγRIIIα binding and ADCC by reducing the fucosylation of IgG. Some strategies involve the development of production cell lines that either eliminate or decrease the expression of α-(1,6) fucosyltransferase. Alternative strategies to reduce fucosylation include silencing the α-(1,6) fucosyltransferase gene by using RNAi. However, enzymatic defucosylation of N-glycans in vitro is still challenging because the N-glycans are embedded between two Fc domains. The presence of the Fc domains restricts the access of α-fucosidase to the fucose residues, resulting in steric hindrance and leading to low efficiency of enzymatic defucosylation.

Only few α-L-fucosidases have been disclosed to be able to remove the core fucose from the innermost GlcNAc residue after removing the external N-glycan on the Fc region, such as α-L-fucosidase from bovine kidney, α-L-fucosidase from human (FucA1), α-L-fucosidase from Bacteroides fragilis (BfFuc), and α-L-fucosidase C from Lactobacillus casei (AlfC). Since the intrinsic function and activity of wild type enzyme are limited and low, developing a modified enzyme to efficiently cleave the core fucoses from N-glycans is needed.

### SUMMARY

In one embodiment, the present disclosure provides a mutant form of α-L-fucosidase for the improved enzymatic hydrolysis of fucose in vitro.

In one embodiment, the present disclosure provides mutant α-L-fucosidase comprising a polypeptide having at least 95% sequence identity to SEQ ID NO: 1 and having a substitution mutation at amino acid position 247 (K247).

In one embodiment, the present disclosure provides mutant α-L-fucosidase comprising a polypeptide having at least 97% sequence identity to SEQ ID NO: 1 and having a substitution mutation at amino acid position 247 (K247).

In one embodiment, the present disclosure provides mutant α-L-fucosidase comprising a polypeptide having at least 99% sequence identity to SEQ ID NO: 1 and having a substitution mutation at amino acid position 247 (K247).

In one embodiment, the present disclosure provides mutant α-L-fucosidase comprising a polypeptide having the sequence of SEQ ID NO: 1 and having a substitution mutation at amino acid position 247 (K247).

In some embodiments, the substitution mutation is selected from the group consisting of K247A, K247C, K247D, K247E, K247F, K247G, K247I, K247L, K247M, K247N, K247P, K247Q, K247S, K247T, K247V, K247W, and K247Y.

In some embodiments, the mutant α-L-fucosidase has the sequence of any one of SEQ ID NOs: 2-18.

In some embodiments, the mutant α-L-fucosidase in present disclosure is useful for the efficient cleavage of core fucose in native glycoproteins without denaturation or functional deterioration of glycoproteins.

In some embodiments, the fucosidase described herein can hydrolyze one or more α-(1,2), α-(1,3), α-(1,4), and α-(1,6)-linked fucoses. The fucoses may be present in N- and/or O-linked glycans in a glycoconjugate.

In some embodiments, the α-fucosidase is a recombinant Bacteroides α-fucosidase.

In some embodiments, the α-fucosidase exhibits pH optimum at 6.5-7.5.

In some embodiments the present disclosure provides a composition comprising the mutant α-L-fucosidase as described above to facilitate the Fc glycoengineering of antibodies or Fc fusion proteins, such as therapeutic antibodies.

In some embodiments, the composition further comprises at least one glycosidase.

In some embodiments, the glycosidase may be an exoglycosidase.

In some embodiments, the glycosidase may be an endoglycosidase. The endoglycosidase includes, but not limited to, Endo-beta-N-acetylglucosaminidases (NAG), EndoA, EndoF1, EndoF2, EndoF3, EndoH, EndoM, EndoS, EndoS2, and variants thereof.

In some embodiments, the composition is useful and efficient for making defucosylation of a glycoconjugate in vitro. Particularly, the composition described herein is useful for making core defucosylation of glycoproteins in vitro.

In some embodiments, the core defucosylation is core α-(1,6) defucosylation.

In some embodiments, the core defucosylation is core α-(1,3) defucosylation.

In some embodiments, the defucosylation can be performed without denaturation or functional deterioration of glycoproteins.

In one embodiment, the present disclosure provides a method of making a defucosylated glycoconjugate in vitro, comprising contacting a glycoconjugate comprising one or more fucoses with an endoglycosidase and the mutant α-L-fucosidase as described above sequentially or simultaneously.

In some embodiments, the method comprises the sequential steps of: (a) contacting the glycoconjugate with the endoglycosidase; and (b) contacting the glycoconjugate with the mutant α-L-fucosidase as described above.

In some embodiments, the method further comprises the step (c) terminating the reaction.

In some embodiments, the step (c) is conducted at about 65° C. for 15-25 minutes.

In some embodiments, the step (a) and/or step (b) is conducted at about 37° C. for 0.5-2 hours.

In some embodiments, the method comprises the sequential step of: (a) contacting the glycoconjugate with the endoglycosidase includes, but not limited to, Endo-beta-N-acetylglucosaminidases (NAG), EndoA, EndoF1, EndoF2, EndoF3, EndoH, EndoM, EndoS, EndoS2, and variants thereof; (b) contacting the glycoconjugate with the mutant α-fucosidase of the invention described above. The glycoconjugate comprises one or more fucoses selected from α-(1,2), α-(1,3), α-(1,4), and α-(1,6)-linked fucoses. The fucoses may be present in N- and/or O-linked glycans in a glycoconjugate.

In some embodiments, the method comprises contacting the composition comprising the mutant α-L-fucosidase described above and at least one glycosidase. The endoglycosidase includes, but not limited to, Endo-beta-N-acetylglucosaminidases (NAG), EndoA, EndoF1, EndoF2, EndoF3, EndoH, EndoM, EndoS, EndoS2, and variants thereof. The glycoconjugate comprises one or more fucoses selected from α-(1,2), α-(1,3), α-(1,4), and α-(1,6)-linked fucoses. The fucoses may be present in N- and/or O-linked glycans in a glycoconjugate.

### DETAILED DESCRIPTION

For convenience, certain terms employed in the context of the present disclosure are collected here. Unless defined otherwise, all technical and scientific terms used herein have the same meaning as commonly understood by one of the ordinary skill in the art to which this disclosure belongs.

The singular forms “a”, “an”, and “the” are used herein to include plural referents unless the context clearly dictates otherwise.

The term “substantial identity” or “substantially identical,” when referring to a polypeptide indicates that, when optimally aligned with appropriate polypeptide insertions or deletions with another amino acid residues, there is polypeptide sequence identity in at least 95%, and more preferably at least 96%, 97%, 98%, or 99% of the amino acid residues to the entire sequence of reference polypeptide sequence as measured by any well-known algorithm of sequence identity, such as BLAST, ALIGN or Megalign (DNASTAR) software. A polypeptide sequence having substantial identity to a reference polypeptide sequence may exhibit the same function.

Ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to +20%, preferably +10%, and more preferable±5%.

As used herein, the term “glycan” refers to a polysaccharide or oligosaccharide. Glycans can be homopolymers or heteropolymers of monosaccharide residues and can be linear or branched. Glycan may also be used to refer to the carbohydrate portion of a glycoconjugate, such as a glycoprotein, glycolipid, or proteoglycan.

As used herein, the term “glycosidase” refers to an enzyme that catalyzes the hydrolysis of the glycosidic linkage or bond.

As used herein, the term “glycoconjugate” refers to all molecules in which at least one sugar moiety is covalently linked to at least one other moiety, including but not limited to N-linked glycoproteins, O-linked glycoproteins, glycolipids, proteoglycans, etc.

As used herein, the term “N-linked glycoproteins” refers to a glycoprotein in which glycans attached to a nitrogen of normally asparagine or arginine side chains.

As used herein, the term “O-linked glycoproteins” refers to a glycoprotein in which glycans attached to the hydroxy oxygen of normally serine, threonine, tyrosine, hydroxylysine, or hydroxyproline side chains, or to oxygens on lipids such as ceramide.

The first aspect of the invention relates to a polypeptide fragment comprising a mutant form of α-L-fucosidase from Lactobacillus casei having the sequence substantial identity to the polypeptide SEQ ID NO: 1 and a substitution mutation at position 247.

SEQ ID NO: 1 refers to the sequence of α-fucosidase from Lactobacillus casei, with the GeneBank accession number of CAQ67984.1.

(SEQ ID NO: 1)
MNDNVAWFKQAKYGMMIHWGLYSLLAGEYRGESSSAYAEWIQSKFQIPN
AEYGNLATAFNPLYFDAKKIVALAKQCGMQYLVVTTKHHDGFAMYHSKV
DAYNVYDATPFHRDIIGELAEACQKAGLKFGLYYSQDLDWHDPNGGGYK
SNDVETAGTTWDNSWDFPDEDQKNFDLCFDNKILPQIKEIMSNYGDIAT
AWFDVPMTLSEAQSQTIYDTVRELQPNCLINSRLGNGKYDFVSLGDNEI
PKNKEDMNKTDVDYNEITGFKPSPLGLYETAGTINDSWGFSYHDQNWKT
PRTLYRYKQHLNDFGINYLLNVGLDPLGRVPMMAEENLLAAKALEDEANR

In some embodiments, the substitution mutation as described above is selected from the group consisting of K247A, K247C, K247D, K247E, K247F, K247G, K247I, K247L, K247M, K247N, K247P, K247Q, K247S, K247T, K247V, K247W, and K247Y, preferably K247D and K247E.

In some embodiments, the substitution mutation as described above is K247D, and the mutant α-L-fucosidase has the sequence of

(SEQ ID NO: 2)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

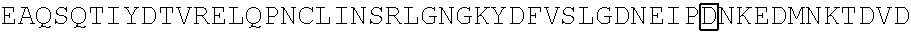

YNEITGFKPSPLGLYETAGTINDSWGFSYHDQNWKTPRTLYRYKQHLNDFGIN
YLLNVGLDPLGRVPMMAEENLLAAKALEDEANR

In some embodiments, the substitution mutation as described above is K247E, and the mutant α-L-fucosidase has the sequence of

(SEQ ID NO: 3)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

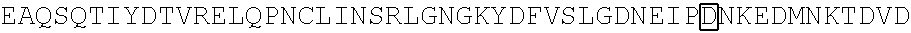

YNEITGFKPSPLGLYETAGTINDSWGFSYHDQNWKTPRTLYRYKQHLNDFGIN
YLLNVGLDPLGRVPMMAEENLLAAKALEDEANR

In some embodiments, the substitution mutation as described above is K247F, and the mutant α-L-fucosidase has the sequence of

(SEQ ID NO: 4)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

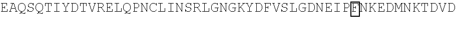

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247A, and the mutant α-L-fucosidase has the sequence of

(SEQ ID NO: 5)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

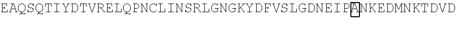

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247C, and the mutant α-L-fucosidase has the sequence of

(SEQ ID NO: 6)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

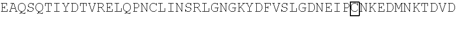

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247G, and the mutant α-L-fucosidase has the sequence of

(SEQ ID NO: 7)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

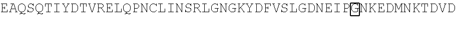

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247I and the mutant α-L-fucosidase has the sequence of

(SEQ ID NO: 8)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

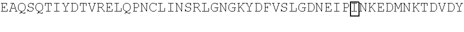

NEITGFKPSPLGLYETAGTINDSWGFSYHDQNWKTPRTLYRYKQHLNDFGINY
LLNVGLDPLGRVPMMAEENLLAAKALEDEANR.

In some embodiments, the substitution mutation as described above is K247L, and the mutant α-L-fucosidase has the sequence of

(SEQ ID NO: 9)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

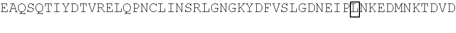

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247M, and the mutant α-L-fucosidase has the sequence of

(SEQ ID NO: 10)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

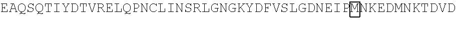

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247N, and the mutant

(SEQ ID NO: 11)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

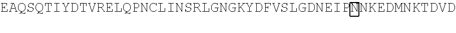

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247P, and the mutant α-L-fucosidase has the sequence of

(SEQ ID NO: 12)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

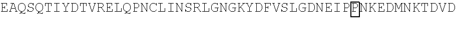

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247Q, and the mutant

(SEQ ID NO: 13)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

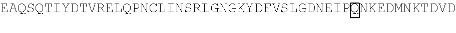

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247S, and the mutant α-L-fucosidase has the sequence of

(SEQ ID NO: 14)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

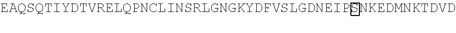

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247T, and the mutant α-L-fucosidase has the sequence of

(SEQ ID NO: 15)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

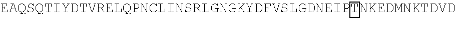

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247V, and the mutant α-L-fucosidase has the sequence of

(SEQ ID NO: 16)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

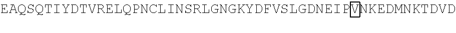

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247W, and the mutant α-L-fucosidase has the sequence of

(SEQ ID NO: 17)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

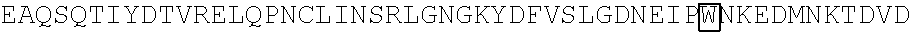

YNEITGFKPSPLGLYETAGTINDSWGFSYHDQNWKTPRTLYRYKQHLNDFGIN
YLLNVGLDPLGRVPMMAEENLLAAKALEDEANR.

In some embodiments, the substitution mutation as described above is K247Y, and the mutant α-L-fucosidase has the sequence of

(SEQ ID NO: 18)
MNDNVAWFKQAKYGMMIHWGLYSLLAGEYRGESSSAYAEWIQSKFQIPNAE
YGNLATAFNPLYFDAKKIVALAKQCGMQYLVVTTKHHDGFAMYHSKVDAYN
VYDATPFHRDIIGELAEACQKAGLKFGLYYSQDLDWHDPNGGGYKSNDVETA
GTTWDNSWDFPDEDQKNFDLCFDNKILPQIKEIMSNYGDIATAWFDVPMTLS

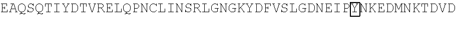

YNEITGFKPSPLGLYETAGTINDSWGFSYHDQNWKTPRTLYRYKQHLNDFGIN
YLLNVGLDPLGRVPMMAEENLLAAKALEDEANR.

The mutant α-fucosidase can hydrolyze one or more α-(1,2), α-(1,3), α-(1,4), and α-(1,6)-linked fucoses. The fucose may be present in N- and/or O-linked glycans in a glycoconjugate. The fucose can be a core α-(1,3) fucose or a core α-(1,6) fucose.

The second aspect of the invention relates to a composition comprising the mutant α-fucosidase described above and at least one glycosidase.

In some embodiments, the glycosidase is an exoglycosidase.

In some embodiments, the glycosidase is an endoglycosidase. The endoglycosidase includes, but not limited to, Endo-beta-N-acetylglucosaminidases (NAG), EndoA, EndoF1, EndoF2, EndoF3, EndoH, EndoM, EndoS, EndoS2, and variants thereof.

In some embodiments, the endoglycosidase is EndoS comprising a mutant at position D233.

In some embodiments, the endoglycosidase is EndoS2 comprising mutant(s) at position T138, D226, T227, and/or T228.

In some embodiments, the endoglycosidase is EndoS2 comprising mutant(s) selected from the group consisting of T138D, T138E, T138F, T138H, T138K, T138L, T138M, T138N, T138Q, T138R, T138V, T138W, D182Q, D226Q, T227Q, and T228Q.

In some embodiments, the composition is useful and efficient for making defucosylation of a glycoconjugate in vitro. Particularly, the composition described herein is useful for making core defucosylation of glycoproteins in vitro. The defucosylation can be performed without denaturation or functional deterioration of glycoproteins.

In some embodiments, the composition of the invention relates to efficient hydrolysis of the N-glycan on the Fc region of the antibody. As used herein, the terms “N-glycan” refers to an N-linked oligosaccharide attached by an N-acetylglucosamine (GlcNAc) linked to the amide nitrogen of an asparagine residue in an Fc region of the antibody or fragment thereof.

In some embodiments, the N-glycan is high mannose, hybrid, or complex types with or without core fucose.

The third aspect of the invention relates to a method of making a defucosylated glycoconjugate in vitro. The glycoconjugate can be treated with the endoglycosidase and the mutant α-fucosidase as described above sequentially or simultaneously.

In some embodiment, the method comprises the step of: (a) contacting the glycoconjugate with the endoglycosidase includes but not limited to Endo-beta-N-acetylglucosaminidases (NAG), EndoA, EndoF1, EndoF2, EndoF3, EndoH, EndoM, EndoS, EndoS2, and variants thereof; (b) contacting the glycoconjugate with the mutant α-fucosidase of the invention described above. The glycoconjugate comprises one or more fucoses selected from α-(1,2), α-(1,3), α-(1,4), and α-(1,6)-linked fucoses. The fucoses may be present in N- and/or O-linked glycans in a glycoconjugate.

In some embodiments, the method comprises contacting the composition comprising the mutant α-L-fucosidase described above and at least one glycosidase. The endoglycosidase includes but not limited to Endo-beta-N-acetylglucosaminidases (NAG), EndoA, EndoF1, EndoF2, EndoF3, EndoH, EndoM, EndoS, EndoS2, and variants thereof. The glycoconjugate comprises one or more fucoses selected from α-(1,2), α-(1,3), α-(1,4), and α-(1,6)-linked fucoses. The fucoses may be present in N- and/or O-linked glycans in a glycoconjugate.

In some embodiments, the endoglycosidase is Streptococcus pyogenes endoglycosidase S2, Streptococcus pyogenes endoglycosidase S having a mutation at amino acid position D233, Streptococcus pyogenes endoglycosidase S2 having a mutation at amino acid position T138, Streptococcus pyogenes endoglycosidase S2 having a mutation at amino acid position D182, Streptococcus pyogenes endoglycosidase S2 having a mutation at amino acid position D184, Streptococcus pyogenes endoglycosidase S2 having a mutation at amino acid position D186, Streptococcus pyogenes endoglycosidase S2 having a mutation at amino acid positionD226, or Streptococcus pyogenes endoglycosidase S2 having a mutation at amino acid position T227.

In some embodiments, the endoglycosidase is EndoS comprising a mutant at position D233.

In some embodiments, the endoglycosidase is EndoS2 comprising mutant(s) at position T138, D226, T227, and/or T228.

In some embodiments, the endoglycosidase is EndoS2 comprising mutant(s) selected from the group consisting of T138D, T138E, T138F, T138H, T138K, T138L, T138M, T138N, T138Q, T138R, T138V, T138W, D182Q, D226Q, T227Q, and T228Q.

In some embodiments, the durations of reaction mixture is set to at least 20 minutes, 30 minutes, 40 minutes, 50 minutes, 60 minutes, 70 minutes, 80 minutes, 90 minutes or 100 minutes, preferably less than 60 minutes. The reaction temperature preferably takes place at room temperature, more preferably at approximately 20° C., 25° C., 30° C., 35° C., 40° C. or 45° C., and most preferably at approximately 37° C.

### Examples

### Example 1. Protein Expression Construct

The wild-type α-fucosidases C (AlfC) were amplified by polymerase chain reaction (PCR) from Lacticaseibacillus casei BL23 genomic DNA (ATCC 393) and cloned into pET47N with His-tag fusion in the N-terminus. Substitution of the wild-type lysine at position 247 with each of the 19 other natural amino acids was generated by saturation mutagenesis using the following forward primers and plasmid encoded wild type AlfC as the template.

TABLE 1
Forward primers used for site-directed mutagenesis.

| Mutation | Forward Primer | SEQ ID NO. |
|---|---|---|
| K247A | 5′-GCG AAC AAA GAA GAT ATG AAC AAG ACC-3′ | 19 |
| K247C | 5′-TGC AAC AAA GAA GAT ATG AAC AAG ACC-3′ | 20 |
| K247D | 5′-GAT AAC AAA GAA GAT ATG AAC AAG ACC-3′ | 21 |
| K247E | 5′-GAA AAC AAA GAA GAT ATG AAC AAG ACC-3′ | 22 |
| K247F | 5′-TTT AAC AAA GAA GAT ATG AAC AAG ACC-3′ | 23 |
| K247G | 5′-GGC AAC AAA GAA GAT ATG AAC AAG ACC-3′ | 24 |
| K247H | 5′-CAT AAC AAA GAA GAT ATG AAC AAG ACC-3′ | 25 |
| K247I | 5′-ATT AAC AAA GAA GAT ATG AAC AAG ACC-3′ | 26 |
| K247L | 5′-CTG AAC AAA GAA GAT ATG AAC AAG ACC-3′ | 27 |
| K247M | 5′-ATG AAC AAA GAA GAT ATG AAC AAG ACC-3′ | 28 |
| K247N | 5′-AAC AAC AAA GAA GAT ATG AAC AAG ACC-3′ | 29 |
| K247P | 5′-CCG AAC AAA GAA GAT ATG AAC AAG ACC-3′ | 30 |
| K247Q | 5′-CAG AAC AAA GAA GAT ATG AAC AAG ACC-3′ | 31 |
| K247R | 5′-CGT AAC AAA GAA GAT ATG AAC AAG ACC-3′ | 32 |
| K247S | 5′-AGC AAC AAA GAA GAT ATG AAC AAG ACC-3′ | 33 |
| K247T | 5′-ACC AAC AAA GAA GAT ATG AAC AAG ACC-3′ | 34 |
| K247V | 5′-GTG AAC AAA GAA GAT ATG AAC AAG ACC-3′ | 35 |
| K247W | 5′-TGG AAC AAA GAA GAT ATG AAC AAG ACC-3′ | 36 |
| K247Y | 5′-TAT AAC AAA GAA GAT ATG AAC AAG ACC-3′ | 37 |

All substitutions used the following as the reverse primer: 5′-CGG GAT CTC GTT ATC GCC CAG-3′ (SEQ ID NO: 38). The PCR program used for saturation mutagenesis was as follows: 95° C. for 3 minutes, 16 cycles of 95° C. for 45 seconds, 58° C. for 50 seconds, 68° C. for 8 minutes, with a final step of 72° C. for 10 minutes. Mutations were confirmed by DNA sequencing. Other enzymes used in the study such as EndoS or EndoS2 were cloned into pET28a with His-tag fusion in N-terminus.

### Example 2. Protein Expression and Purification

Protein expression constructs were transformed into BL21 (DE3) (EMD Biosciences, San Diego, CA). Protein was overexpressed by 0.1 mM isopropyl β-D-thiogalactopyranoside (IPTG) induction at 16° C. for 20 hours. Cells were disrupted by high-pressure homogenizer, followed by collecting the supernatant after centrifugation. The collected supernatant was loaded into Ni-NTA agarose column and washed with ten folds of column volume of washing buffer (Tris-HCl, pH 7.4, 200 mM sodium chloride, and 40 mM imidazole). Elution was conducted by two folds of column volume of elution buffer (Tris-HCl, pH 7.4, 200 mM sodium chloride, and 300 mM imidazole). Protein purity was examined by SDS-PAGE, and the protein buffer was exchanged to reaction buffer (50 mM Tris-HCl, 50 mM mannitol, 50 mM sorbitol, pH 7.0) by Amicon® Ultra-0.5 Centrifugal Filter Devices (UFC5010BK, 10 kDa cut-off).

### Example 3. Core Defucosylation of Glycoproteins

The core defucosylation activity of mutant fucosidase was performed by analyzing the core fucose percentage after enzyme treatment. Trastuzumab (Herceptin®) (24 mg) was treated with EndoS2 (15.2 μg) at 37° C. for 1 hour, followed by reacting with the AlfC fucosidase mutant (2.8 μg) at 37° C. for additional 1 hour. The reaction was suspended by heat-inactivation at 65° C. for 20 minutes and washed by ddH₂O in an Amicon® Ultra filter devices for 3 times. The supernatant was collected for mass analysis (Table 2). The percentage of core fucose on the N-glycan in the blank group (No enzyme) is about 87%. The defucoslyation activity of wild type AlfC enzyme is about 19%. Mutation on the K247 residue to negative charge amino acid residue significantly improve the defucosylation activity to about 80%, while other mutants on the K247 also exhibit enhanced defucosylation activity excepting positive charge residues. The defucosylation percentage is calculated by

$$\frac{Experimantial N \% - Blank N \%}{Blank NF \%}$$

TABLE 2
Analysis of defocusylation percentage.

| | Analyses of Fucose by Heavy Chain Mass Intensity | | |
|---|---|---|---|
| | N | NF | De-fucosylation Percentage |
| Enzyme | (%) | (%) | (%) |
| No enzyme | 12.57 | 87.43 | 0 |
| AlfC | 29.79 | 70.21 | 19.7 |
| AlfC^{K247D} | 83.60 | 16.40 | 81.2 |
| AlfC^{K247E} | 81.72 | 18.28 | 79.1 |
| AlfC^{K247S} | 53.26 | 46.74 | 46.5 |
| AlfC^{K247R} | 19.06 | 80.94 | 7.4 |
| AlfC^{K247H} | 31.35 | 68.65 | 21.5 |
| AlfC^{K247T} | 54.43 | 45.57 | 47.9 |
| AlfC^{K247N} | 43.94 | 56.06 | 35.9 |
| AlfC^{K247Q} | 55.58 | 44.42 | 49.2 |
| AlfC^{K247G} | 66.38 | 33.62 | 61.5 |
| AlfC^{K247P} | 71.91 | 28.09 | 67.9 |
| AlfC^{K247A} | 63.10 | 36.90 | 57.8 |
| AlfC^{K247V} | 61.48 | 38.52 | 55.9 |
| AlfC^{K247I} | 66.33 | 33.67 | 61.5 |
| AlfC^{K247L} | 69.07 | 30.93 | 64.6 |
| AlfC^{K247M} | 61.34 | 38.66 | 55.8 |
| AlfC^{K247F} | 79.83 | 20.17 | 76.9 |
| AlfC^{K247Y} | 68.19 | 31.81 | 63.6 |
| AlfC^{K247C} | 56.20 | 43.80 | 49.9 |
| AlfC^{K247W} | 64.18 | 35.82 | 59.0 |

“NF” represents the structure of core GlcNAc-fucose and “N” represents the structure of core GlcNAc.

While the present disclosure has been described in conjunction with the specific embodiments set forth above, many alternatives thereto and modifications and variations thereof will be apparent to those of ordinary skill in the art. All such alternatives, modifications, and variations are regarded as falling within the scope of the present disclosure.

### REFERENCES

U.S. Pat. No. 11,193,155B2
Klontz E H, Li C, Kihn K, Fields J K, Beckett D, Snyder G A, Wintrode P L, Deredge D, Wang L X, Sundberg E J. Structure and dynamics of an α-fucosidase reveal a mechanism for highly efficient IgG transfucosylation. Nat Commun. 2020 Dec. 4; 11 (1): 6204
Li C, Zhu S, Ma C, Wang L X. Designer α1,6-Fucosidase Mutants Enable Direct Core Fucosylation of Intact N-Glycopeptides and N-Glycoproteins. J Am Chem Soc. 2017 Oct. 25; 139 (42): 15074-15087.
Osanjo G, Dion M, Drone J, Solleux C, Tran V, Rabiller C, Tellier C. Directed evolution of the alpha-L-fucosidase from Thermotoga maritima into an alpha-L-transfucosidase. Biochemistry. 2007 Jan. 30; 46 (4): 1022-33.
   U.S. Pat. No. 10,415,021

The invention claimed is:

1. A mutant α-L-fucosidase comprising a polypeptide having at least 95% sequence identity to SEQ ID NO: 1 and having a substitution mutation at amino acid position 247 (K247).

2. The mutant α-L-fucosidase of claim 1, wherein the substitution mutation is selected from the group consisting of K247A, K247C, K247D, K247E, K247F, K247G, K247I, K247L, K247M, K247N, K247P, K247Q, K247S, K247T, K247V, K247W, and K247Y.

3. The mutant α-L-fucosidase of claim 1, having the sequence of any one of SEQ ID NOs: 2-18.

4. A composition comprising the mutant α-L-fucosidase according to claim 3 and at least one glycosidase.

5. A method of making a defucosylated glycoconjugate in vitro, comprising contacting a glycoconjugate comprising one or more fucoses with an endoglycosidase and the mutant α-L-fucosidase according to claim 1 sequentially or simultaneously.

6. The method of claim 5, comprising the sequential steps of:
(a) contacting the glycoconjugate with the endoglycosidase; and
(b) contacting the glycoconjugate with the mutant α-L-fucosidase.

7. The method of claim 6, further comprising (c) terminating the reaction.

8. The method of claim 7, wherein the step (c) is conducted at about 65° C. for 15-25 minutes.

9. The method of any-exe of claims 6-8, wherein the step (a) and/or step (b) is conducted at about 37° C. for 0.5-2 hours.

10. The method of claim 6, wherein the endoglycosidase is Endo-beta-N-acetylglucosaminidases (NAG), EndoA, EndoF1, EndoF2, EndoF3, EndoH, EndoM, EndoS, EndoS2, and variants thereof.

11. The method of claim 10, wherein EndoS2 comprises a substitution mutation selected from the group consisting of T138D, T138E, T138F, T138H, T138K, T138L, T138M, T138N, T138Q, T138R, T138V, T138W, D182Q, D226Q, T227Q, and T228Q.

12. The method of claim 5, wherein the fucoses are selected from the group consisting of α-(1,2), α-(1,3), α-(1,4), and α-(1,6)-linked fucoses.


SEQUENCE LISTING (38 sequences)

SEQ ID NO: 1 - protein, 344 aa, Lactobacillus casei
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 2 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPDNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 3 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPENKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 4 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPFNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 5 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPANKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 6 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPCNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 7 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPGNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 8 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPINKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 9 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPLNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 10 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPMNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 11 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPNNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 12 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPPNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 13 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPQNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 14 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPSNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 15 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPTNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 16 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPVNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 17 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPWNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 18 - protein, 344 aa, unidentified
  source 1..344: note = Artificial Sequence
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPYNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 19 - DNA, 27 bp, unidentified
  source 1..27: mol_type = other DNA; note = Artificial Sequence
gcgaacaaagaagatatgaacaagacc

SEQ ID NO: 20 - DNA, 27 bp, unidentified
  source 1..27: mol_type = other DNA; note = Artificial Sequence
tgcaacaaagaagatatgaacaagacc

SEQ ID NO: 21 - DNA, 27 bp, unidentified
  source 1..27: mol_type = other DNA; note = Artificial Sequence
gataacaaagaagatatgaacaagacc

SEQ ID NO: 22 - DNA, 27 bp, unidentified
  source 1..27: mol_type = other DNA; note = Artificial Sequence
gaaaacaaagaagatatgaacaagacc

SEQ ID NO: 23 - DNA, 27 bp, unidentified
  source 1..27: mol_type = other DNA; note = Artificial Sequence
tttaacaaagaagatatgaacaagacc

SEQ ID NO: 24 - DNA, 27 bp, unidentified
  source 1..27: mol_type = other DNA; note = Artificial Sequence
ggcaacaaagaagatatgaacaagacc

SEQ ID NO: 25 - DNA, 27 bp, unidentified
  source 1..27: mol_type = other DNA; note = Artificial Sequence
cataacaaagaagatatgaacaagacc

SEQ ID NO: 26 - DNA, 27 bp, unidentified
  source 1..27: mol_type = other DNA; note = Artificial Sequence
attaacaaagaagatatgaacaagacc

SEQ ID NO: 27 - DNA, 27 bp, unidentified
  source 1..27: mol_type = other DNA; note = Artificial Sequence
ctgaacaaagaagatatgaacaagacc

SEQ ID NO: 28 - DNA, 27 bp, unidentified
  source 1..27: mol_type = other DNA; note = Artificial Sequence
atgaacaaagaagatatgaacaagacc

SEQ ID NO: 29 - DNA, 27 bp, unidentified
  source 1..27: mol_type = other DNA; note = Artificial Sequence
aacaacaaagaagatatgaacaagacc

SEQ ID NO: 30 - DNA, 27 bp, unidentified
  source 1..27: mol_type = other DNA; note = Artificial Sequence
ccgaacaaagaagatatgaacaagacc

SEQ ID NO: 31 - DNA, 27 bp, unidentified
  source 1..27: mol_type = other DNA; note = Artificial Sequence
cagaacaaagaagatatgaacaagacc

SEQ ID NO: 32 - DNA, 27 bp, unidentified
  source 1..27: mol_type = other DNA; note = Artificial Sequence
cgtaacaaagaagatatgaacaagacc

SEQ ID NO: 33 - DNA, 27 bp, unidentified
  source 1..27: mol_type = other DNA; note = Artificial Sequence
agcaacaaagaagatatgaacaagacc

SEQ ID NO: 34 - DNA, 27 bp, unidentified
  source 1..27: mol_type = other DNA; note = Artificial Sequence
accaacaaagaagatatgaacaagacc

SEQ ID NO: 35 - DNA, 27 bp, unidentified
  source 1..27: mol_type = other DNA; note = Artificial Sequence
gtgaacaaagaagatatgaacaagacc

SEQ ID NO: 36 - DNA, 27 bp, unidentified
  source 1..27: mol_type = other DNA; note = Artificial Sequence
tggaacaaagaagatatgaacaagacc

SEQ ID NO: 37 - DNA, 27 bp, unidentified
  source 1..27: mol_type = other DNA; note = Artificial Sequence
tataacaaagaagatatgaacaagacc

SEQ ID NO: 38 - DNA, 21 bp, unidentified
  source 1..21: mol_type = other DNA; note = Artificial Sequence
cgggatctcgttatcgcccag